(19) United States
(12) Patent Application Publication
(10) Pub. No.: US 2012/0215274 A1
(43) Pub. Date: Aug. 23, 2012

(54) ACCELEROMETER ENHANCED IMPLANTABLE CARDIO-DEVICE

(75) Inventors: Steve Koh, South Pasadena, CA (US); Gene A. Bornzin, Simi Valley, CA (US)

(73) Assignee: PACESETTER, INC., Sylmar, CA (US)

(21) Appl. No.: 13/032,509

(22) Filed: Feb. 22, 2011

(51) Int. Cl. A61N 1/365

(52) U.S. Cl. 607/17

(57) ABSTRACT

An implantable medical device (“IMD”) processes and analyzes valuable clinical information regarding cardiac performance. A database or correlator is pre-customized to the specific patient, by correlating signals received by a remote accelerometer associated with heart movements with accurate heart sounds recorded from a microphone to provide a more effective and customized basis for estimating heart sound. The information is then used to better control an implantable medical device.

### FIELD

The present disclosure relates to implantable medical devices (IMDs). More particularly, the disclosed embodiments relate to accurately determining heart sounds with an accelerometer enhanced IMD.

### BACKGROUND

An implantable medical device (IMD), such as a pacemaker and/or implantable cardioverter-defibrillator (ICD), regulates or synchronizes the beating of the heart with electrical impulses, delivered by electrical leads having electrodes contacting the heart muscles. Some IMDs include a number of different sensors and logic allowing them to monitor the rate and rhythm of the heart as well as to measure various cardiac surrogates that provide information on the operation of the heart. One such sensor is an accelerometer. Accelerometers are microelectronic devices that can send a signal to be received remotely based upon movement, such as of a specific desired region of the heart muscle.

Heart rate can be monitored in a variety of ways. One type of monitoring is based on heart sounds. A clinically accepted heart sound originates from the left ventricle wall. Therefore, an upper chest located IMD with an accelerometer receives a signal that differs from that received by a microphone near the fourth ventricular region (V4). The difference results from various distorting influences, including the distance between the accelerometer and the V4 region and the fact that the heart sound is dampened by organs. Thus, prior accelerometer approaches to measuring heart rates based upon heart sounds attempted to filter out noise created by the abdominal wall, the lung and other organs; or to sub-divide cardiac signals for pre-processing prior to use with an IMD. These approaches have yielded unsatisfactory and/or inaccurate results. Still, an IMD based or related accelerometer signal contains valuable clinical information regarding cardiac performance but usually with a delay and/or distorted amplitude compared to the clinically accepted V4 heart sound. It would be desirable to determine with greater accuracy the rhythms of the heart, based on heart sound data observed at any installed IMD.

### SUMMARY

In one embodiment, a controller for an IMD is customized for each particular patient. In this embodiment, signals from a remote accelerometer are pre-measured. These signals are indicative of that patient's heart vibrations, especially over a variety of states (such as resting, walking, sleeping, reading, talking, and the like). A correlator correlates or corrects these signals with actually recorded heart sounds indicative of the same respective heart movements over the same states. The correlator may be in the form of a database, a logic device (including, but not limited to programmable logic and/or memory devices, and the like) and/or a circuit (including, but not limited to an adaptive equalization filter, or the like). Recorded heart sounds may be obtained by a microphone (e.g., a stethoscope), or the like, placed in proximity to the patient's heart; the microphone may be removed after the heart sounds have been received, or the microphone may be left with the patient. Once the heart sounds have been correlated with the remote accelerometer signals, the controller may better control an IMD as the correlated/corrected accelerometer signals more accurately measure an actual heart movement, or lack of heart movement (as during an arrhythmia or heart stoppage).

Another embodiment of this disclosure shows the use of an accelerometer placed near but not at (hereafter referred to as remote) the patient's heart, for example, but not limited to placement on or implanted within the upper chest of a patient, or on a leg, abdomen or other location. By monitoring and comparing the accelerometer measurement with the actual heart sound, a table, formula, circuitry, adaptive logic, database (database) or other correlation (hereinafter generally referred to as a database or correlation) is developed to determine actual heart sound for the particular patient based on prior determined heart sound/accelerometer correlations. In one embodiment, the accelerometer measured signals, which may be inaccurate, may be corrected or enhanced by more accurate heart sound values to produce a correlation for accelerometer measured signals such that later measured accelerometer signals can be more accurately considered as indicative of specific actual heart movement, or lack of heart movement. The correlation may be used to observe, diagnose or treat a patient, including for the control of an IMD.

Some embodiments of this disclosure teach how to transform an accelerometer signal into an effectively measured V4 heart sound, by using a modeling scheme referred to herein as an adaptive equalizer. An equalization (EQ) filter, usually adjustable, may compensate for the unequal frequency response of some other signal processing circuit or system. An EQ filter allows the user to adjust one or more parameters that determine the overall shape of the filter's transfer function. Such an EQ filter may modify or compensate the accelerometer measured signal to determine what the heart sound is or would have been. This compensated or correlated accelerometer signal (hereafter generally referred to as a correlated accelerometer signal) is a better measure to use in the controlling an IMD than an un-compensated or un-correlated accelerometer signal.

In some embodiments, a customized or semi-customized database or correlator is developed for each patient by measuring and storing or otherwise correlating heart sound and accelerometer signals for a variety of states, especially at the beginning of a monitoring or operational period. Such signal may also be enhanced by use of an EQ filter in various embodiments. The correlator or database may then be used to control an IMD for better patient management.

In some embodiments, such as a clinical situation, a microphone embedded wand may be used to collect accelerometer and heart sound simultaneously. A computer may compute EQ filter coefficients and download the coefficients to the IMD. In some embodiments, such as a home situation, a home unit such as Merlin.net is adapted to act in a similar manner. Re-computation of EQ coefficients may be warranted if atrioventricular delay changes or ischemia becomes evident.

In some embodiments, newly measured or later acquired accelerometer values (with or without additional correlation with newly measured or later acquired heart sound), hereafter referred to as performance accelerometer values, may be used to develop, modify, expand, correct and/or update the correlator or correlated accelerometer signals which may then be used in the control of an IMD.

According to an aspect of the present disclosure, a method controls an implantable medical device (IMD). The method includes obtaining estimated heart sounds based on observed accelerometer signals and a relationship between training signals from a remote accelerometer indicative of movements of a heart and pre-recorded heart sounds indicative of the same heart movements. The method also includes controlling the implantable medical device, at least in part, based on the estimated heart sounds.

In another aspect, a system controls an implantable medical device. The system includes an accelerometer and a controller. The accelerometer is adapted to be located on a patient remote from a patient heart and further adapted to generate a signal based on measured heart movements of the patient. The controller is adapted to control the implantable medical device at least partially in response to an estimated heart sound derived from a relationship between the accelerometer signal and previously recorded heart sounds.

In yet another aspect, a system controls an implantable medical device (IMD). The system includes obtaining means and controlling means. The obtaining means obtains estimated heart sounds based on observed accelerometer signals and a relationship between training signals from a remote accelerometer indicative of a movements of a heart and pre-recorded heart sounds indicative of the same heart movements. The controlling means controls the implantable medical device, at least in part, based on the estimated heart sounds.

In a still further aspect, a computer readable medium tangibly stores code for controlling an implantable medical device (IMD). The code includes code to obtain estimated heart sounds based on observed accelerometer signals and a relationship between training signals from a remote accelerometer indicative of movements of a heart and pre-recorded heart sounds indicative of the same heart movements. The code also includes code to control the implantable medical device, at least in part, based on the estimated heart sounds.

The foregoing has outlined rather broadly the features and technical advantages of the present teachings in order that the detailed description of the teachings that follows may be better understood. Additional features and advantages of the teachings will be described hereinafter which form the subject of the claims of the teachings. It should be appreciated by those skilled in the art that the conception and specific embodiment disclosed may be readily utilized as a basis for modifying or designing other structures for carrying out the same purposes of the present teachings. It should also be realized by those skilled in the art that such equivalent constructions do not depart from the spirit and scope of the teachings as set forth in the appended claims. The novel features which are believed to be characteristic of the teachings, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present teachings.

### BRIEF DESCRIPTION OF FIGURES

For a more complete understanding of the present disclosure, reference is now made to the following descriptions taken in conjunction with the accompanying drawings.

The following description includes the best mode or modes presently contemplated for practicing the present teachings. The description is not to be taken in a limiting sense but is merely for the purpose of describing the general principles of the illustrative embodiments. The scope of the present teachings should be ascertained with reference to the claims. In the description that follows, like numerals or reference designators will refer to like parts or elements throughout.

For simplicity and clarity of illustration, elements shown in the figures have not necessarily been drawn to scale. For example, the dimensions of some of the elements may be exaggerated relative to other elements for clarity of presentation. Furthermore, reference numerals may be repeated among the figures to indicate corresponding or analogous elements.

FIG. 1 schematically illustrates an exemplary IMD in electrical communication with the heart of a patient

FIG. 2 schematically illustrates an exemplary implantable stimulation device configured as a system in which the various embodiments of the present teachings may operate.

FIG. 3 is a flowchart of a process for correlating heart sound with accelerometer signals to produce a database or correlator.

FIG. 4 is a flowchart of a process for correcting, enhancing or otherwise correlating accelerometer signals with accurate heart measurements, as may be produced by heart sounds to obtain a more accurate heart measure within a margin of error.

FIG. 5 is a graph showing measured accelerometer signals transformed into simulated heart sounds or corrected or correlated accelerometer signals, along with a graph of measured heart sound.

FIGS. 6A-B are schematics of an adaptive equalization filter which may be used to correlate accelerometer signals with heart sound, or with prior correlated accelerometer values.

FIG. 7 is a schematic of an adaptive equalization filter which may be used to correlate accelerometer signals with heart sound, or with prior correlated accelerometer values.

### DETAILED DESCRIPTION

Historically and empirically, a body mounted accelerometer (such as located in an IMD) signal is different from auscultation received heart sounds. Some embodiments of this disclosure teach how to transform an accelerometer signal into an effectively measured heart sound, such as a V4 heart sound, by using a modeling scheme referred to herein as an adaptive equalizer.

In the following detailed description, numerous specific details are set forth in order to provide a thorough understanding of some embodiments. However, it will be understood by persons of ordinary skill in the art that some embodiments may be practiced without these specific details. In other instances, well-known methods, procedures, components, units and/or circuits have not been described in detail so as not to obscure the discussion. The following description includes the best mode presently contemplated for practicing the present teachings. The description is not to be taken in a limiting sense but is merely for the purpose of describing the general principles of the illustrative embodiments. The scope of the present teachings should be ascertained with reference to the claims. In the description that follows, like numerals or reference designators will refer to like parts or elements throughout.

Some portions of the following detailed description are presented in terms of algorithms and symbolic representations of operations on data bits or binary digital signals within a computer memory. These algorithmic descriptions and representations may be the techniques used by those skilled in the data processing arts to convey the substance of their work to others skilled in the art.

An algorithm is here, and generally, considered to be a self-consistent sequence of acts or operations leading to a desired result. These include physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers or the like. It should be understood, however, that all of these and similar terms are to be associated with the appropriate physical quantities and are merely convenient labels applied to these quantities.

Discussions herein utilizing terms such as, for example, “processing”, “computing”, “calculating”, “determining”, “establishing”, “analyzing”, “checking”, or the like, may refer to operation(s) and/or process(es) of a computer, a computing platform, a computing system, or other electronic computing device, that manipulate and/or transform data represented as physical (such as, electronic) quantities within the computer's registers and/or memories into other data similarly represented as physical quantities within the computer's registers and/or memories or other information storage medium that may store instructions to perform operations and/or processes.

The terms “plurality” and “a plurality” as used herein includes, for example, “multiple” or “two or more”. For example, “a plurality of items” includes two or more items.

Unless otherwise noted, or as may be evident from the context of their usage, any terms, abbreviations, acronyms or scientific symbols and notations used herein are to be given their ordinary meaning in the technical discipline to which the disclosure most nearly pertains. The following or above-noted terms, abbreviations and/or acronyms may be used throughout the descriptions presented herein and should generally be given the described meaning(s) unless contradicted or elaborated upon by other descriptions set forth herein. Some of the terms set forth below may be registered trademarks (®).

When terms (such as abbreviations) are used in the description, no distinction should be made between the use of capital (uppercase) and lowercase letters. For example “ABC”, “abc” and “Abc”, or any other combination of upper and lower case letters with these 3 letters in the same order should be considered to have the same meaning as one another, unless indicated or explicitly stated to be otherwise. The same commonality generally applies to glossary or other introduced terms (such as abbreviations), which include subscripts, which may appear with or without subscripts, such as “Xyz” and “X_{yz}”. Additionally, plurals of glossary or other introduced terms may or may not include an apostrophe before the final “s”—for example, ABCs or ABC's.

Disclosed herein is an embodiment for a controller for an IMD which is customized, or semi-customized, for each particular patient. One such embodiment would be achieved by pre-measuring signals received at a remote accelerometer which are indicative of that patient's heart movements, especially over a variety of states (such as resting, walking, sleeping, reading, talking, and the like), and then correlating or correcting those signals with heart sounds indicative of the same respective heart movements over the same states. The correlator may be in the form of a database, a logic device (including, but not limited to programmable logic and/or memory devices, and the like) and/or a circuit (including, but not limited to an adaptive equalization filter, or the like). Heart sounds may be obtained by a microphone, or the like, placed in proximity to the patient's heart; the microphone may be removed after the heart sounds have been received, or the microphone may be left with the patient. Once the heart sounds have been correlated with the remote accelerometer signals, the controller may be used to better control an IMD as the correlated/corrected accelerometer signals may more accurately measure an actual heart movement.

In some embodiments, newly measured or later acquired accelerometer values (with or without additional correlation with newly measured or later acquired heart sound), hereafter referred to as performance accelerometer values, may be used to develop, modify, expand, correct and/or update the correlator or correlated accelerometer signals which may then be used in the control of an IMD.

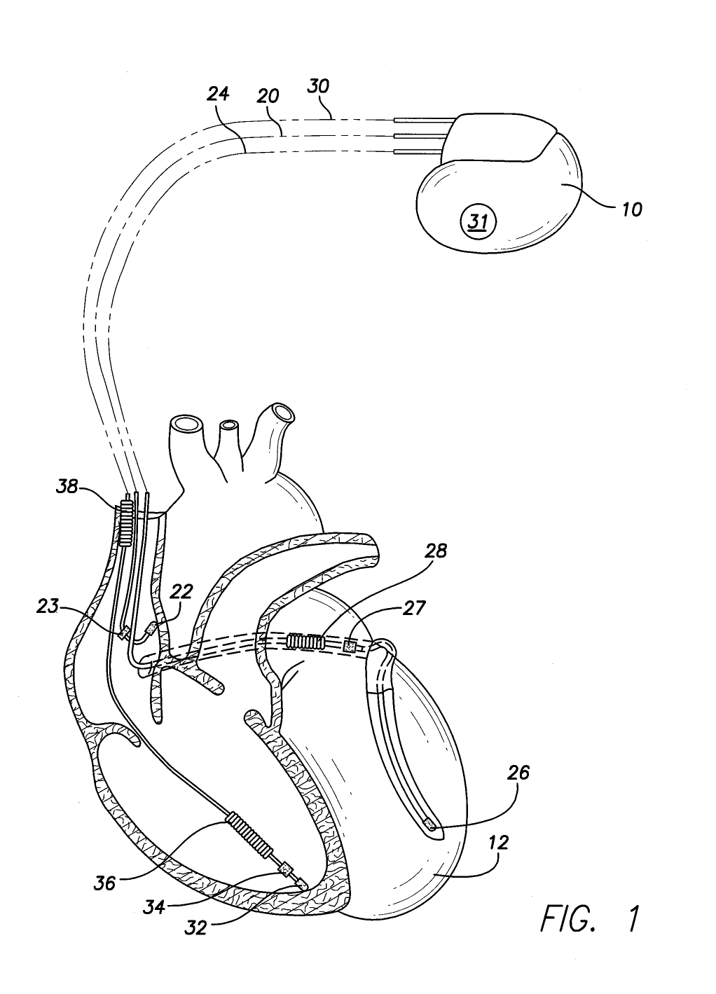

With reference to FIG. 1, an exemplary IMD will be described in detail. The IMD 10 is in electrical communication with the heart 12 of a patient by way of three leads, 20, 24 and 30, suitable for delivering multi-chamber stimulation and shock therapy. To sense atrial cardiac signals and to provide right atrial chamber stimulation therapy, the IMD 10 is coupled to an implantable right atrial lead 20 having at least an atrial tip electrode 22, which typically is implanted in the right atrial appendage, and an atrial ring electrode 23.

To sense left atrial and ventricular cardiac signals and to provide left chamber pacing therapy, the IMD 10 is coupled to a “coronary sinus” lead 24 designed for placement in the “coronary sinus region” via the coronary sinus or for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus. Accordingly, an exemplary coronary sinus lead 24 is designed to receive atrial and ventricular cardiac signals and to deliver left ventricular pacing therapy using at least a left ventricular tip electrode 26, left atrial pacing therapy using at least a left atrial ring electrode 27, and shocking therapy using at least a left atrial coil electrode 28.

The IMD 10 is also shown in electrical communication with the heart by way of an implantable right ventricular lead 30 having, in this embodiment, a right ventricular tip electrode 32, a right ventricular ring electrode 34, a right ventricular (RV) coil electrode 36, and a superior vena cava (SVC) coil electrode 38. Typically, the right ventricular lead 30 is transvenously inserted into the heart so as to place the right ventricular tip electrode 32 in the right ventricular apex so the RV coil electrode 36 is positioned in the right ventricle and the SVC coil electrode 38 is positioned in the superior vena cava. Accordingly, the right ventricular lead 30 is capable of receiving cardiac signals, and delivering stimulation in the form of pacing and shock therapy to the right ventricle. To provide a “vibratory alert” signal (from a motor with an offset mass that can be provided in the device can), an additional electrode can be provided in proximity to the device can. An accelerometer 31 can also be provided.

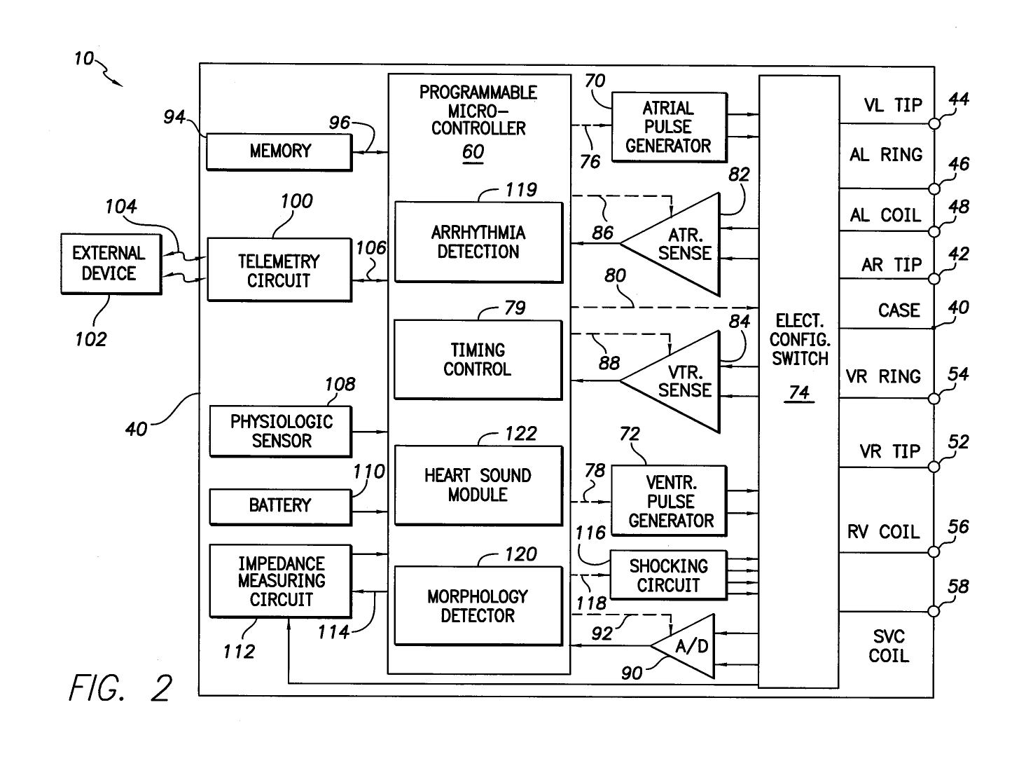

As illustrated in FIG. 2, a simplified block diagram is shown of the multi-chamber implantable IMD 10, which is capable of treating both fast and slow arrhythmias with stimulation therapy, including cardioversion, defibrillation, and pacing stimulation. The IMD 10 is configured as a system in which the various embodiments of the present teachings may operate. While a particular multi-chamber device is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation.

The housing 40 for the IMD 10, shown schematically in FIG. 2, is often referred to as the “can”, “case” or “case electrode” and may be programmably selected to act as the return electrode for all “unipolar” modes. The housing 40 may further be used as a return electrode alone or in combination with one or more of the coil electrodes, 28, 36 and 38, (FIG. 1) for shocking purposes. The housing 40 further includes a connector (not shown) having multiple terminals, 42, 44, 46, 48, 52, 54, 56 and 58 (shown schematically and, for convenience, the names of the electrodes to which they are connected are shown next to the terminals).

As such, to achieve right atrial sensing and pacing, the connector includes at least a right atrial tip terminal (AR TIP) 42 adapted for connection to the atrial tip electrode 22 (FIG. 1) and a right atrial ring (AR RING) electrode (not shown) adapted for connection to the right atrial ring electrode 23 (FIG. 1). To achieve left chamber sensing, pacing and shocking, the connector includes at least a left ventricular tip terminal (VL TIP) 44, a left atrial ring terminal (AL RING) 46, and a left atrial shocking terminal (AL COIL) 48, which are adapted for connection to the left ventricular ring electrode 26 (FIG. 1), the left atrial tip electrode 27 (FIG. 1), and the left atrial coil electrode 28 (FIG. 1), respectively. To support right chamber sensing, pacing and shocking, the connector further includes a right ventricular tip terminal (VR TIP) 52, a right ventricular ring terminal (VR RING) 54, a right ventricular shocking terminal (RV COIL) 56, and an SVC shocking terminal (SVC COIL) 58, which are adapted for connection to the right ventricular tip electrode 32 (FIG. 1), right ventricular ring electrode 34 (FIG. 1), the RV coil electrode 36 (FIG. 1), and the SVC coil electrode 38 (FIG. 1), respectively. To provide the “vibratory alert” signal, a vibratory alert unit (not shown) generates a signal for an additional terminal (not shown) for connection to the vibratory alert electrode. In one embodiment, the vibratory alert will alert the patient, and then a home monitor can be used to transfer the information associated with the alert from the device 10 to an attending medical professional, who can take the appropriate clinical action.

The IMD 10 includes a programmable microcontroller 60, which controls the various modes of stimulation therapy. As is well known in the art, the microcontroller 60 (also referred to as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 60 includes the ability to process or monitor input signals (data) as controlled by program code stored in a designated block of the memory. The details of the design and operation of the microcontroller 60 are not critical to the present teachings. Rather, any suitable microcontroller 60 may be used that carries out the functions described. The use of microprocessor-based control circuits for performing timing and data analysis functions is well known in the art.

As shown in FIG. 2, an atrial pulse generator 70 and a ventricular pulse generator 72 generate pacing stimulation pulses for delivery by the right atrial lead 20 (FIG. 1), the right ventricular lead 30 (FIG. 1), and/or the coronary sinus lead 24 (FIG. 1) via an electrode configuration switch 74. It is understood that in order to provide stimulation therapy in each of the four chambers of the heart, the atrial and ventricular pulse generators, 70 and 72, may include dedicated, independent pulse generators, multiplexed pulse generators or shared pulse generators. The pulse generators, 70 and 72, are controlled by the microcontroller 60 via appropriate control signals, 76 and 78, respectively, to trigger or inhibit the stimulation pulses.

The microcontroller 60 further includes timing control circuitry 79 that controls the timing of such stimulation pulses (such as pacing rate, atrioventricular (AV) delay, atrial interconduction (A-A) delay, or ventricular interconduction (V-V) delay, and the like) as well as to keep track of the timing of refractory periods, blanking intervals, noise detection windows, evoked response windows, alert intervals, marker channel timing, etc., as is well known in the art. A switch 74 includes multiple switches for connecting the desired electrodes to the appropriate I/O circuits, thereby providing complete electrode programmability. Accordingly, the switch 74, in response to a control signal 80 from the microcontroller 60, determines the polarity of the stimulation pulses (such as unipolar, bipolar, combipolar, etc.) by selectively closing the appropriate combination of switches (not shown) as is known in the art.

Atrial sensing circuits 82 and ventricular sensing circuits 84 may also be selectively coupled to the right atrial lead 20 (FIG. 1), the coronary sinus lead 24 (FIG. 1), and the right ventricular lead 30 (FIG. 1), through the switch 74 for detecting the presence of cardiac activity in each of the four chambers of the heart. Accordingly, the atrial (ATR. SENSE) and ventricular (VTR. SENSE) sensing circuits, 82 and 84, may include dedicated sense amplifiers, multiplexed amplifiers or shared amplifiers and may receive control signals 86, 88 from the controller 60. The switch 74 determines the “sensing polarity” of the cardiac signal by selectively closing the appropriate switches, as is also known in the art. In this way, the clinician may program the sensing polarity independent of the stimulation polarity. Each sensing circuit, 82 and 84, employs one or more low power, precision amplifiers with programmable gain and/or automatic gain control, band pass filtering, and a threshold detection circuit, as known in the art, to selectively sense the cardiac signal of interest. An automatic gain control enables the device 10 to effectively address the difficult problem of sensing the low amplitude signal characteristics of atrial or ventricular fibrillation. The outputs of the atrial and ventricular sensing circuits, 82 and 84, are connected to the microcontroller 60 which, in turn, are able to trigger or inhibit the atrial and ventricular pulse generators, 70 and 72, respectively, in a demand fashion in response to the absence or presence of cardiac activity in the appropriate chambers of the heart.

For arrhythmia detection, the device 10 utilizes the atrial and ventricular sensing circuits, 82 and 84, to sense cardiac signals to determine whether a rhythm is physiologic or pathologic. As used herein “sensing” is reserved for the noting of an electrical signal, and “detection” is the processing of these sensed signals and noting the presence of an arrhythmia. The timing intervals between sensed events (for example: P-waves, R-waves, and depolarization signals associated with fibrillation which are sometimes referred to as “F-waves” or “Fib-waves”) are then classified by the microcontroller 60 by comparing them to a predefined rate zone limit (for example: bradycardia, normal, low rate VT, high rate VT, and fibrillation rate zones) and various other characteristics (for example: sudden onset, stability, physiologic sensors, and morphology, and the like) in order to determine the type of remedial therapy that is needed (for example: bradycardia pacing, anti-tachycardia pacing, cardioversion shocks or defibrillation shocks, and the like).

Cardiac signals are also applied to the inputs of an analog-to-digital (A/D) data acquisition system 90. The data acquisition system 90 is configured to acquire intra-cardiac electrogram (IEGM) signals, convert the raw analog data into a digital signal, and store the digital signals for later processing and/or telemetric transmission to an external device 102. The data acquisition system 90 is coupled to the right atrial lead 20 (FIG. 1), the coronary sinus lead 24 (FIG. 1), and the right ventricular lead 30 (FIG. 1) through the switch 74 to sample cardiac signals across any pair of desired electrodes. The controller 60 controls the data acquisition system via control signals 92.

The microcontroller 60 is further coupled to a memory 94 by a suitable data/address bus 96. The programmable operating parameters used by the microcontroller 60 are stored and modified, as required, in order to customize the operation of the IMD 10 to suit the needs of a particular patient. The memory 94 includes software modules, such as a heart sound correlator, which, when executed or used by the microcontroller 60, provide the operational functions of the implantable IMD 10. Additional operating parameters and code stored on the memory 94 define, for example, pacing pulse amplitude or magnitude, pulse duration, electrode polarity, rate, sensitivity, automatic features, arrhythmia detection criteria, and the amplitude, wave shape and vector of each shocking pulse to be delivered to the patient's heart within each respective tier of therapy. Other pacing parameters include base rate, rest rate and circadian base rate.

Advantageously, the operating parameters of the implantable device 10 may be non-invasively programmed into the memory 94 through a telemetry circuit 100 in telemetric communication with the external device 102, such as a programmer, trans-telephonic transceiver, a diagnostic system analyzer, or even a cellular telephone. The telemetry circuit 100 is activated by the microcontroller by a control signal 106. The telemetry circuit 100 advantageously allows intra-cardiac electrograms and status information relating to the operation of the device 10 (as contained in the microcontroller 60 or memory 94) to be sent to the external device 102 through an established communication link 104. In one embodiment, the IMD 10 further includes a physiologic sensor 108, commonly referred to as a “rate-responsive” sensor because it adjusts pacing stimulation rate according to the exercise state of the patient. However, the physiological sensor 108 may further be used to detect changes in cardiac output, changes in the physiological condition of the heart, or diurnal changes in activity (for example, detecting sleep and wake states). Accordingly, the microcontroller 60 responds by adjusting the various pacing parameters (such as rate, AV Delay, V-V Delay, and others) at which the atrial and ventricular pulse generators, 70 and 72, generate stimulation pulses. While shown as being included within the IMD 10, it is to be understood that the physiologic sensor 108 may also be external to the IMD 10, yet still be implanted within or carried by the patient.

The IMD 10 additionally includes a battery 110, which provides operating power to all of the circuits shown in FIG. 2. For the IMD 10, which employs shocking therapy, the battery 110 is capable of operating at low current drains for long periods of time, and is capable of providing high-current pulses (for example, for capacitor charging) when the patient requires a shock pulse. The battery 110 also has a predictable discharge characteristic so that elective replacement time can be detected. In one embodiment, the device 10 employs lithium/silver vanadium oxide batteries. As further shown in FIG. 2, the device 10 has an impedance measuring circuit 112 enabled by the microcontroller 60 via a control signal 114.

The IMD 10 detects the occurrence of an arrhythmia and automatically applies an appropriate electrical shock therapy to the heart aimed at terminating the detected arrhythmia. To this end, the microcontroller 60 further controls a shocking circuit 116 by way of a control signal 118. The shocking circuit 116 generates shocking pulses of low (up to 0.5 joules), moderate (0.5-10 joules), or high energy (11 to 40 or more joules), as controlled by the microcontroller 60. Such shocking pulses are applied to the heart 12 through at least two shocking electrodes, and as shown in this embodiment, selected from the left atrial coil electrode 28 (FIG. 1), the RV coil electrode 36 (FIG. 1), and/or the SVC coil electrode 38 (FIG. 1). As noted above, the housing 40 may function as an active electrode in combination with the RV coil electrode 36 (FIG. 1), or as part of a split electrical vector using the SVC coil electrode 38 (FIG. 1) or the left atrial coil electrode 28 (FIG. 1) (for example, by using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (such as corresponding to thresholds in the range of 5-40 joules), delivered asynchronously (since R-waves may be too disorganized), and pertaining exclusively to the treatment of fibrillation. Accordingly, the microcontroller 60 is capable of controlling the synchronous or asynchronous delivery of the shocking pulses.

The microcontroller 60 includes a morphology detector 120 for tracking various morphological features within electrical cardiac signals, including intervals between polarization events, elevations between polarization events, durations of polarization events and amplitudes of polarization events. The microcontroller 60 also includes an arrhythmia detection control 119 that analyzes the sensed electrical signals to determine whether or not arrhythmia is being experienced. A heart sound module 122, in cooperation with the memory 94, processes accelerometer signals received from the accelerometer 31 (FIG. 1) to provide accurate heart sound information.

The remaining figures, flow charts, graphs and other diagrams illustrate the operation and novel features of the IMD 10 as configured in accordance with exemplary embodiments of the present teachings. In the flow chart, the various process steps are summarized in individual “blocks.” Such blocks describe specific actions or decisions made or carried out as the process proceeds. Where a microcontroller (or equivalent) is employed, the flow chart provides the basis for heart sound processing that may be used by such a microcontroller (or equivalent IMD controller) to adaptively determine accurate heart sounds. Those skilled in the art may readily write such a program based on the flow chart and other descriptions presented herein.

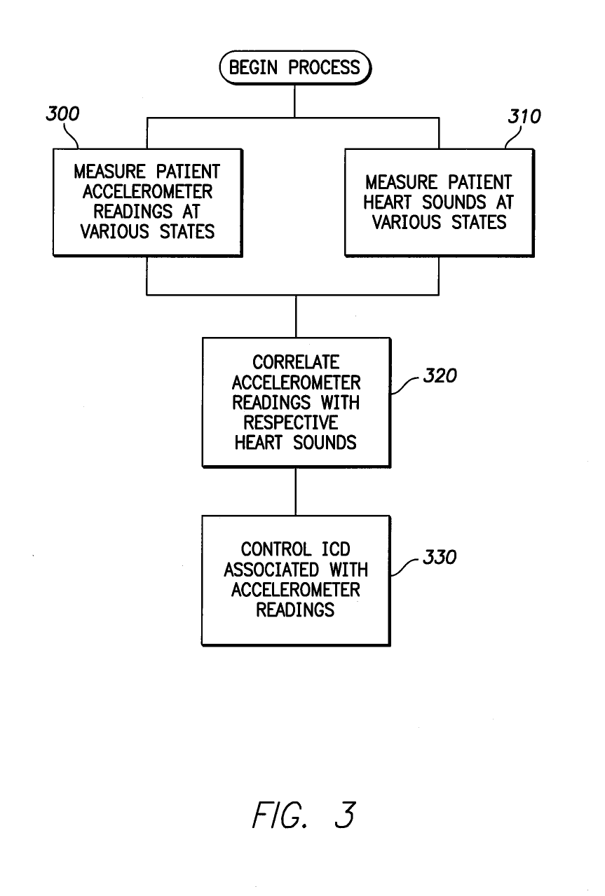

Referring now to FIG. 3, a process for correlating accelerometer signals used in the control of an IMD is described. Initially, at block 300 patient accelerometer readings are measured at various states. This may occur to initialize operation of the IMD or to modify, expand, correct and/or update the correlation. The readings may be stored in the memory 94 in order to control the IMD. At block 310, patient heart sounds are measured at various states. The heart sounds may be recorded prior to implantation to initialize the device 10 or may be periodically recorded at subsequent checkups to update the device. The measured patient heart sounds correspond to specific respective measured patient accelerometer readings. In one embodiment, the pre-recorded heart sounds are recorded simultaneously with the accelerometer data of block 300 for approximately 10 seconds. At block 320 accelerometer readings are correlated with respective heart sounds. At block 330, the IMD is controlled in accordance with the correlated accelerometer readings.

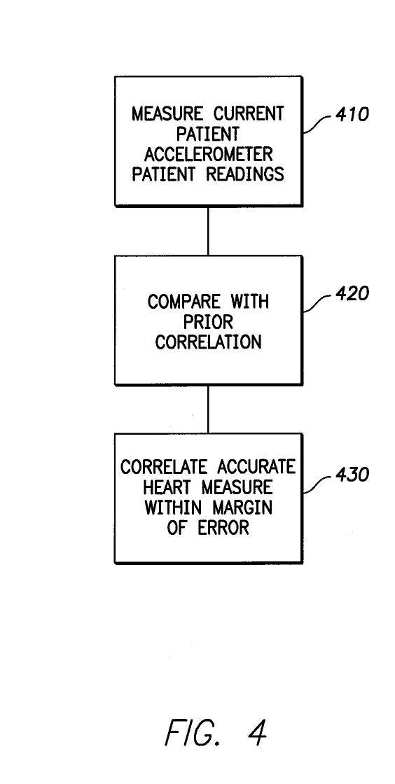

Referring now to FIG. 4, a process to develop, modify, expand, correct and/or update the correlator or correlated accelerometer signals used in the control of an IMD is described. At block 410 current patient accelerometer readings are measured at various states, referring to a second or subsequent set of readings, not the initial patient accelerometer readings. At block 420 the correlation is compared with a prior correlation, such that the new readings will either replace the old readings in certain embodiments, or the new readings will enhance (such as adding additional values, correcting values, and the like). At block 430, an accurate heart measure is correlated within a margin of error. Such measure may include a margin of error, but is a better measure of actual heart movement, or lack of heart movement, than un-correlated accelerometer readings.

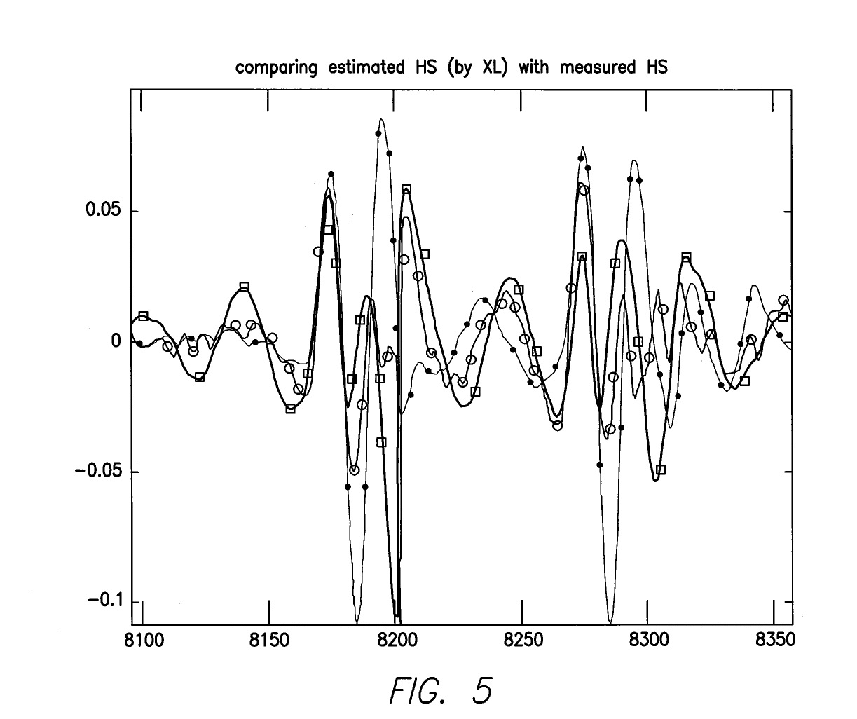

FIG. 5, is a graph showing one example of measured accelerometer data transformed into simulated heart sounds, along with a graph of measured heart sound. A numeral 510, designated with dots, for purposes of clarity, shows an example of a measured accelerometer signal. Numeral 520, designated with squares for purposes of clarity, shows an example of a measured heart sound signal. Numeral 530 designated with circles, for purposes of clarity, shows an example of a correlated accelerometer signal, which is more in-line, phase and amplitude with the measured heart sound, thus a better measure of actual heart movement. The correlation of accelerometer with heart sound may be done in any manner known to one of skill in the art, such as by the use of an adaptive equalization filter described above, and further described below.

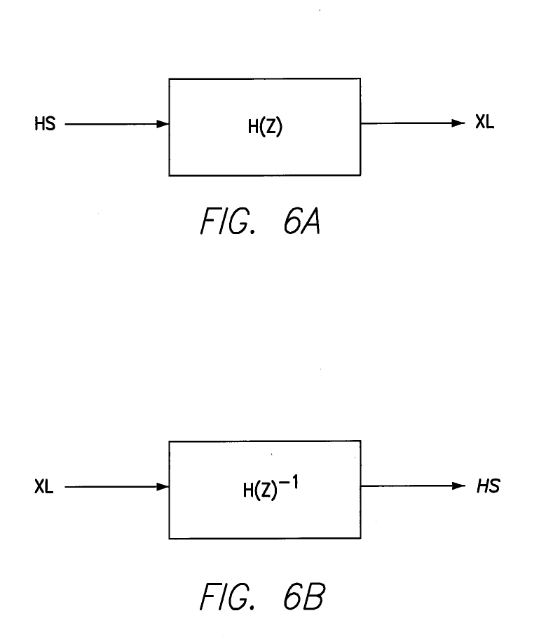

FIGS. 6A-B are schematics of an adaptive equalization filter which may be used to correlate accelerometer data with heart sound data, or with prior correlated accelerometer values. In FIG. 6A, having values generally associated with known adaptive equalization filters, the input HS will be the heart sound data and the output XL will be accelerometer signals or values taken during the interrogation or learning phase, usually before the IMD is activated but may be after IMD activation if performance accelerometer values will be measured, correlated or used. The function H(z) includes at least one coefficient, and can be for example an autoregressive model, that correlates the accelerometer data XL and the heart sound data HS.

After the function H(z) is derived an inverse function H(z)⁻¹ is then applied, as shown if FIG. 6B. The inverse function H(z)⁻¹ is applied to input accelerometer data XL to obtain an estimated heart sound HS. The estimated heart sound HS is compared to the actual heart sound HS. If the difference, E, is small enough, the inverse function H(z)⁻¹ can be used to convert accelerometer data into accurate estimated heart sound data.

In this case, the inverse function H(z)⁻¹ is loaded into, or otherwise transferred to the controller for the IMD. Once the IMD controller has received the inverse function H(z)⁻¹, the coefficients may be used by the controller to transform the accelerometer data into more accurate heart sound data to control the operation of the IMD. Employing coefficients rather than a database is optional, as either will permit the controller to more accurately measure heart movement. In some embodiments, different coefficients are provided based on a state of the patient, for example, walking, exercising, resting, etc.

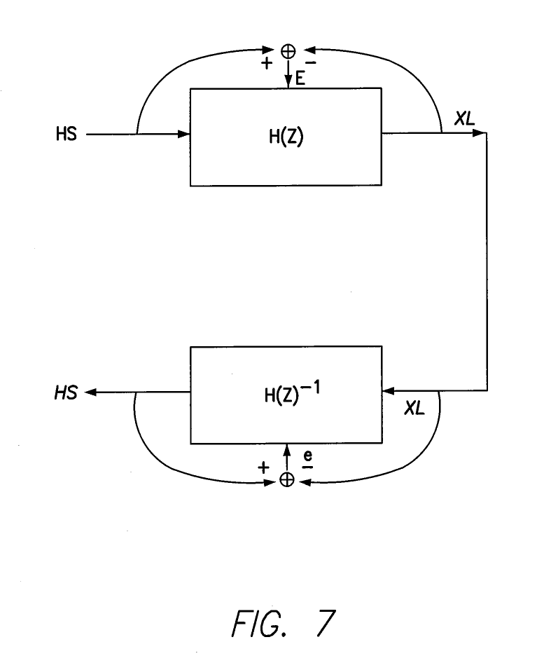

As seen in FIG. 7, a forward+inverse model can be used. Heart sound data HS is input to the autoregressive model H(Z) to obtain estimated accelerometer data XL. An error, E, is minimized to adjust the function H(z). The error, E, is the absolute value of the difference between the actual heart sound HS and the estimated heart sound HS.

The estimated accelerometer data XL is input to the inverse function H(z)⁻¹ to obtain the estimated heart sound data HS. The error, e, is also minimized in an attempt to improve the accuracy of the estimated heart sound data HS. The error, e, is the absolute value of the difference between the model input and the output. In one embodiment, the model is based on well known Kalman filtering.

The methodologies described herein may be implemented by various means depending upon the application. For example, these methodologies may be implemented in hardware, firmware, software, or any combination thereof. For a hardware implementation, the processing units, including programmable microcontroller 60 (FIG. 2) may be implemented within one or more application specific integrated circuits (ASICs), digital signal processors (DSPs), digital signal processing devices (DSPDs), programmable logic devices (PLDs), field programmable gate arrays (FPGAs), processors, controllers, microcontrollers, microprocessors, electronic devices, other electronic units designed to perform the functions described herein, or a combination thereof.

For a firmware and/or software implementation, the methodologies may be implemented with modules (e.g., procedures, functions, and so on) that perform the functions described herein. Any machine or computer readable medium tangibly embodying instructions that may be in a form implantable or coupled to an implantable medical device may be used in implementing the methodologies described herein. For example, software code may be stored in a memory and executed by a processor. When executed by the processor, the executing software code generates the operational environment that implements the various methodologies and functionalities of the different aspects of the teachings presented herein. Memory may be implemented within the processor or external to the processor. As used herein the term “memory” refers to any type of long term, short term, volatile, nonvolatile, or other memory and is not to be limited to any particular type of memory or number of memories, or type of media upon which memory is stored.

The machine or computer readable medium that stores the software code defining the methodologies and functions described herein includes physical computer storage media. A storage medium may be any available medium that can be accessed by the processor of an implantable medical device. By way of example, and not limitation, such computer-readable media can comprise RAM, ROM, EEPROM, CD-ROM or other optical disk storage, magnetic disk storage or other magnetic storage devices, or any other medium that can be used to store desired program code in the form of instructions or data structures and that can be accessed by a computer. As used herein, disk and/or disc includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer readable media.

Although the present teachings and its advantages have been described in detail, it should be understood that various changes, substitutions and alterations can be made herein without departing from the spirit and scope of the present teachings as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present teachings, processes, machines, manufacture, compositions of matter, means, methods, or steps, presently existing or later to be developed that perform substantially the same function or achieve substantially the same result as the corresponding embodiments described herein may be utilized according to the present teachings. Accordingly, the appended claims are intended to include within their scope such processes, machines, manufacture, compositions of matter, means, methods, or steps.

What is claimed is:

1. A method for controlling an implantable medical device (IMD) comprising:
obtaining estimated heart sounds based on observed accelerometer signals and a relationship between training signals from a remote accelerometer indicative of a plurality of movements of a heart and pre-recorded heart sounds indicative of the same heart movements; and
controlling the implantable medical device, at least in part, based on the estimated heart sounds.

2. The method of claim 1, further comprising receiving a plurality of training signals from the remote accelerometer at a plurality of patient states; and
correlating the training accelerometer signals with pre-recorded heart sounds at the same patient states to determine a relationship between the training accelerometer signals and the heart sounds at each patient state.

3. The method according to claim 2 wherein at least some of the plurality of training signals from the remote accelerometer are obtained prior to operation of the implantable medical device.

4. The method according to claim 2 wherein at least some of the plurality of training signals from the remote accelerometer are obtained after initiating operation of the implantable medical device.

5. The method according to claim 2 wherein at least some of the plurality of training signals from the remote accelerometer are performance accelerometer values.

6. The method according to claim 2, in which the pre-recorded heart sounds are recorded simultaneously with the training accelerometer signals.

7. The method according to claim 1 wherein the estimated heart sounds are contained, at least in-part, in a database.

8. The method according to claim 1 further comprising defining the relationship, at least in-part, in at least one mathematical model.

9. A system for controlling an implantable medical device, comprising:
an accelerometer adapted to be located on a patient remote from a patient heart and further adapted to generate a signal based on measured heart movements of the patient; and
a controller adapted to control the implantable medical device at least partially in response to an estimated heart sound derived from a relationship between the accelerometer signal and previously recorded heart sounds.

10. The system of claim 9, further comprising:
a processor configured to receive a plurality of training signals from the remote accelerometer at a plurality of patient states; and
a correlator configured to correlate the training accelerometer signals with pre-recorded heart sounds at the same patient states to determine a relationship between the training accelerometer signals and the heart sounds at each patient state.

11. The system according to claim 10 in which at least some of the plurality of training signals from the remote accelerometer are obtained prior to operation of the implantable medical device.

12. The system according to claim 11 in which at least some of the plurality of training signals from the remote accelerometer are obtained after initiating operation of the implantable medical device.

13. The system according to claim 11 in which at least some of the plurality of training signals from the remote accelerometer are performance accelerometer values.

14. The system according to claim 11, in which the pre-recorded heart sounds are recorded simultaneously with the training accelerometer signals.

15. The system according to claim 10 in which the estimated heart sounds are contained, at least in-part, in a database.

16. The system according to claim 10 in which the processor is further configured to define the relationship, at least in-part, in at least one mathematical model.

17. A system for controlling an implantable medical device (IMD) comprising:
means for obtaining estimated heart sounds based on observed accelerometer signals and a relationship between training signals from a remote accelerometer indicative of a plurality of movements of a heart and pre-recorded heart sounds indicative of the same heart movements; and
means controlling the implantable medical device, at least in part, based on the estimated heart sounds.

18. The system of claim 17, further comprising:
means for receiving a plurality of training signals from the remote accelerometer at a plurality of patient states; and
means for correlating the training accelerometer signals with pre-recorded heart sounds at the same patient states to determine a relationship between the training accelerometer signals and the heart sounds at each patient state.

19. The system according to claim 18, in which the pre-recorded heart sounds are recorded simultaneously with the training accelerometer signals.

20. The system according to claim 17 in which the estimated heart sounds are contained, at least in-part, in a database.
